(19) United States
(12) Patent Application Publication
(10) Pub. No.: US 2018/0303396 A1
(43) Pub. Date: Oct. 25, 2018

(54) A SYSTEM AND A METHOD FOR GNERATING A PROFILE OF STRESS LEVELS AND STRESS RESILIENCE LEVELS IN A POPULATION

(71) Applicant: GLOBAL STRESS INDEX PTY LTD, Sydney, New South Wales (AU)

(72) Inventors: Travis Leigh Wild, Sydney (AU); Stephen Aaron Foster, Sydney (AU)

(21) Appl. No.: 15/525,879

(22) PCT Filed: Nov. 11, 2015
(86) PCT No.: PCT/AU2015/050704

§ 371 Date: May 10, 2017

(30) Foreign Application Priority Data
Nov. 11, 2014 (AU) 2014904524

(51) Int. Cl. A61B 5/16; A61B 5/00; G16H 10/20; G16H 50/30; G16H 10/60

(52) U.S. Cl. CPC A61B 5/165; A61B 5/7275; G16H 10/60; G16H 50/30; G16H 10/20

(57) ABSTRACT

A method and a system for generating stress level information indicative of a stress level of a plurality of individuals. The method and system comprises receiving, via a network, individual stress information for each of the plurality of individuals, and generating, in a processing system, a statistical value for the stress level of the plurality of individuals by statistically processing the individual stress information for each of the plurality of individuals.

### CROSS REFERENCE TO RELATED APPLICATIONS

This application claims the benefit of international application PCT/AU2015/050704 with an international filing date of Nov. 11, 2015 which claims priority from application 2014904524 filed in Australia on Nov. 11, 2014, the disclosures of both of which are specifically incorporated herein by reference.

### TECHNICAL FIELD

The disclosure herein generally relates to a system and a method for generating a profile of stress levels and stress resilience levels in a population of people.

### BACKGROUND

Stress is believed to contribute to a range of diseases such as heart disease, obesity, diabetes and cancer. Stress is also believed to adversely influence the productivity of workers. One estimate is that the cost to employers of worker absenteeism and presenteeism alone is US$2,500 per worker in developed countries every year. The combined cost of stress-related health care expenses and lost productivity is in the many of billions of dollars every year.

Stress in humans can be categorised as either acute (short-term) or chronic (long-term). Examples of sources of acute stress include physical activities to which the individual is not accustomed, an upset in a relationship, a bereavement, public speaking, or having a higher than usual workload for days, weeks or months. People normally adapt to acute stress and then recover from it as soon as the stress passes. Because of this ability to adapt and recover, acute stress per se may not be as damaging to our wellbeing as chronic stress.

However, stress resilience can be an indication of underlying damage occurring to a person's wellbeing. Stress resilience is a person's ability to respond to an acute stress event or an acute stress state. For example, one particularly important aspect of stress level resilience is the time taken for the individual acute stress elements and indicators, either singular or in combination, to return to ‘unstressed’ or baseline levels following any particular stressful event.

As an example, if a person becomes acutely stressed—exercising or giving a presentation at work—their stress indicators such as heart rate, blood pressure, sweat (skin conductivity) and so on, would elevate. These stress measures can be detected and recorded.

When the stress subsides, these indicators should return to their previous baseline over the next 15 to 30 minutes. However, in a person with ‘diminishing stress resilience levels’, their stress response can be more accelerated (more ‘excitable’), can be heightened or accentuated (more ‘reactive’), and take longer to return to ‘normal’ with their stress ‘half-life’ or ‘resolution to baseline’ taking longer (slower resolution). The more rapid and accentuated the response and the longer the recovery time, the less stress resilience the individual has, even if their stress measures do eventually return to ‘normal’ or ‘baseline’ levels.

When looking at a large group of people, assessing the stress levels in a population for example, it is very useful to determine the underlying stress characteristics, or stress profile of the population to provide a better context for analyzing the particular stress levels or characteristics of any one individual of the population. This can greatly increase the accuracy and efficacy of assessing acute and chronic stress in an individual.

There is a need for detailed data on stress in large numbers of people. Governments would be able to use this data in a number of ways. Firstly, detailed stress data would enable governments and other organizations to objectively assess the benefit of stress management methods and programs.

Secondly, it would be economically beneficial for a government to be able to rapidly determine the impact of their policies on the stress experienced by the people it governs.

Almost any government policy has the potential to affect the levels of stress experienced by the people it governs, and the stress will in turn have an impact on the productivity of the economy. Unfortunately there is no way to directly and rapidly measure the impact of policy decisions on stress experienced by populations.

One of the issues hampering research into stress is an inability to quickly measure stress in large numbers of people, such as populations of cities or countries. Current methods of measuring stress in people generally comprise either psychometric testing, physiological testing or cognitive function testing. However, testing large numbers of people involves performing these kinds of tests on a massive scale, which is slow, labour intensive and expensive.

The expense of doing stress testing has led to relatively small numbers of people being included in research studies. The only option is to extrapolate trends from a small test group of people, but the process assumes the sample group is representative of the entire population, which is unlikely, and it is difficult to find a sample group of people who are willing to give up their time to be tested on a regular basis.

### SUMMARY

In an embodiment there is a method for generating stress level information indicative of a stress level of a plurality of individuals, the method comprising: receiving, via a network, individual stress information for each of the plurality of individuals; and generating, in a processing system, a statistical value for the stress level of the plurality of individuals by statistically processing the individual stress information for each of the plurality of individuals.

In an embodiment, the method comprises the steps of receiving, via the network, personal information for each of a plurality of persons and individual stress information for each of the plurality of persons in the processing system, using personal information for each of the plurality of persons to select from the plurality of persons the plurality of individuals.

In an embodiment, the personal information comprises at least one of date of birth information, place of birth information, gender information, ethnicity information, occupation information, postcode information, education information, health insurance coverage information, relationship status information, number of children information, pet information, exercise habit information, eating habit information, health history information, and information indicative of stress management methods currently being used.

In an embodiment, the method comprises the step of receiving, via the network, information indicative of at least one of a stress modifying circumstance and stress modifying event and correlating a stress feature in the statistical measure with the at least one of the stress modifying circumstance and the stress modifying event.

In an embodiment, the stress feature comprises a change in the statistical value of the stress level of the plurality of individuals.

In an embodiment, the stress modifying circumstance and the stress modifying event comprises at least one of: internet keyword search behaviour information, content information, sentiment or topics of social media communications information, date information, time information, public holiday information, temperature information, humidity information, weather information, traffic information, news information, current affairs information, consumer purchasing information, financial market information, economic information, announcement information, political event information, sporting event information, topical event information, home loan interest rate information, housing information, employment information, survey information, poll information, voting schedule information, business confidence information, business investment information, and business productivity information.

In an embodiment, the method comprises the step of generating, in the processing system, a stress index using the statistical value.

In an embodiment, the method comprises the step of the processing system sending the stress index to a plurality of computing devices.

In an embodiment, the step of the processing system sending the statistical measure of the stress level of the plurality of individuals to the plurality of computing devices.

In an embodiment, the individual stress information for each of the plurality of individuals comprises at least one of psychometric information for each of the plurality of individuals, physiological information for each of the plurality of individuals, behavioural information for each of the plurality of individuals, and cognitive function information for each of the plurality of individuals.

In an embodiment, the individual stress information for each of the plurality of individuals comprises at least two of psychometric information for each of the plurality of individuals, physiological information for each of the plurality of individuals, behavioural information for each of the plurality of individuals, and cognitive function information for each of the plurality of individuals.

In an embodiment, the stress information for each of the plurality of individuals comprises the psychometric information for each of the plurality of individuals.

In an embodiment, the method comprises the step of generating the psychometric information for each of the plurality of individuals by each of the plurality of individuals responding to an electronic stress questionnaire.

In an embodiment, the psychometric information for each of the plurality of individuals is indicative of a plurality of chronic stress indicators for the each of the plurality of individuals.

In an embodiment, the stress information for each of the plurality of individuals comprises the physiological information for each of the plurality of individuals.

In an embodiment, the method comprises the step of generating the physiological information for each of the plurality of individuals.

In an embodiment, the step of generating the physiological information for each of the plurality of individuals comprises the step of generating information for each of a plurality of physiological functions in each of the plurality of individuals.

In an embodiment, the step of generating the physiological information for each of the plurality of individuals comprises the step of generating at least one of heart rate information, heart rate variability information, respiratory rate information, respiratory rate variability information, blood pressure information, physical movement information, cortisol level information, skin conductivity information, skin temperature information, blood oxygen saturation information, surface electromyography information, electroencephalography information, blood information, saliva information, skin conductance information, information regarding the chemicals found on or within the skin, and urine information.

In an embodiment, the stress information for each of the plurality of individuals comprises behavioural information for each of the plurality of individuals.

In an embodiment, the method comprises the step of generating the behavioural information for each of the plurality of individuals.

In an embodiment, the step of generating the behavioural information for each of the plurality of individuals comprises at least one of the steps of: generating eye movement information indicative of eye movement of each of the plurality of individuals; generating location information indicative of a plurality of locations each of the plurality of individuals has been; generating nearby device information indicative of the nearby presence a plurality of devices of a plurality of people to each of the plurality of individuals; generating internet browsing history information for each of the plurality of individuals; generating keystroke rate, cadence, typing style, pressure or ‘force’ detection information for the individual; generating voice analysis, including tone, cadence, word and phrase detection information for the individual; generating telephone usage analysis, including call time, numbers dialed and time of day calls placed information for the individual; generating driving style, including steering inputs, acceleration, deceleration, braking, speed of driving, brake and accelerator force and data from door pressure sensor information for the individual; generating movement, body temperature, television usage, including channels watched, time watched and eye movement whilst watching, refrigerator analytics, heating and cooling analytics information for the individual; generating bicycle data, including pedal force, pedaling cadence, acceleration, speed, routes taken, GPS data, altimeter data, time on bicycle, pedometer data information for the individual; generating pedometer data and gait analysis information for the individual; generating application usage information indicative of application usage by each of the plurality of individuals; generating media consumption information indicative of media consumption by each of the plurality of individuals; generating spending behaviour information indicative of the spending behaviour of each of the plurality of individuals; generating food choice information indicative of a plurality of food choices made by each of the plurality of individuals; generating social outing information indicative of social outing activity of each of the plurality of individuals; and generating leave information indicative of leave taken by each of the plurality of individuals.

In an embodiment, the stress information for each of the plurality of individuals comprises the cognitive function information for each of the plurality of individuals.

In an embodiment, the method comprises the step of generating the cognitive function information for each of the plurality of individuals.

In an embodiment, the step of generating the cognitive function information for each of the plurality of individuals comprises at least one of the steps of: generating memory function information indicative of a memory function of each of the plurality of individuals; generating reaction time information indicative of a reaction time of each of the plurality of individuals; generating attention ability, peripheral vision and comprehension ability of the individual; and generating decision-making ability information indicative of a decision-making ability of each of the plurality of individuals.

In an embodiment, the method comprises the step of generating a stress resilience score indicative of each of the plurality of individuals response to acute stress. Preferably, the stress resilience score is indicative of one or more of the time taken for the plurality of individuals to respond to an acute stress event, if the plurality of individuals exhibit any response to an acute stress event, and if so, the level of response exhibited by the plurality of individuals to an acute stress event and the time taken for the plurality of individuals' stress information to return to baseline levels following a period of acute stress.

In another embodiment, there is a processing system for generating stress level information indicative of a stress level of a plurality of individuals, the system comprising: a receiver configured to receive via a network individual stress information for each of the plurality of individuals; and a statistical value generator configured to generate a statistical value for the stress level of the plurality of individuals by statistically processing the individual stress information for each of the plurality of individuals.

### BRIEF DESCRIPTION OF THE FIGURES

Embodiments will now be described by way of example only with reference to the accompanying figures in which:

FIG. 1 shows a block diagram of the components of the architecture of the system and a method for generating a profile of stress levels and stress resilience levels in a population.

### DESCRIPTION OF EMBODIMENTS

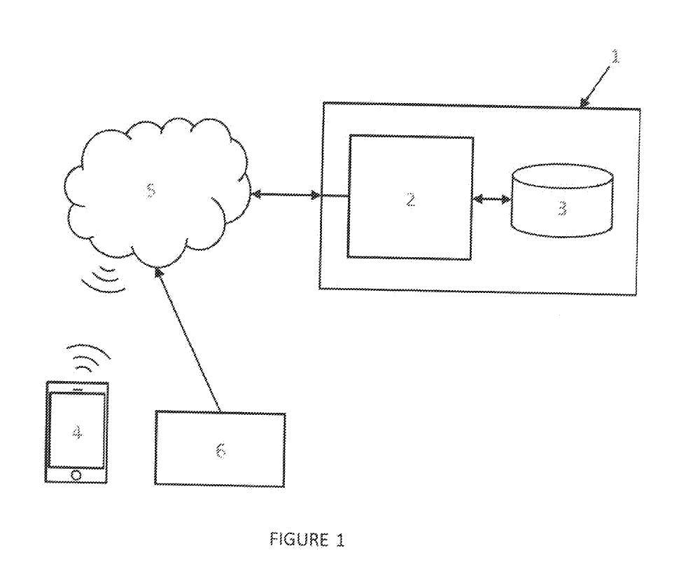

FIG. 1 is a block diagram of the components of the architecture of the stress profiler, which includes:
1. population stress profiler
2. server
3. database
4. personal stress profiler
5. communication network
6. general data source.

The population stress profiler (1) includes a computer server (2) in communication with a database (3).

The computer server (2) is configured to execute the steps of an embodiment of a method described herein. The method may be coded in a program for instructing the processor of the computer server. The program is, in this embodiment stored in the non-volatile memory, but could be stored in FLASH, EPROM or any other form of tangible media within or external of the computer server. The program generally, but not necessarily, comprises a plurality of software modules that cooperate when installed on the system so that the steps of an embodiment of the method are performed. The software modules, at least in part, correspond to the steps of the method or components of the system described herein. The functions or components may be compartmentalised into modules or may be fragmented across several software and/or hardware modules. The software modules may be formed using any suitable language, examples of which include C++ and assembly. The program may take the form of an application program interface or any other suitable software structure.

The computer system coupled with the computer server (2) includes a suitable microprocessor such as, or similar to, the INTEL XEON or AMD OPTERON micro processor connected over a bus 16 to memory which includes a suitable form of random access memory 18 of around 1 GB, or generally any suitable alternative capacity, and a non-volatile memory 20 such as a hard disk drive or solid state non-volatile memory (e.g. NAND-based FLASH memory) having a capacity of around 500 Gb, or any alternative suitable capacity. Alternative logic devices may be used in place of the microprocessor. Examples of suitable alternative logic devices include application-specific integrated circuits, field programmable gate arrays (FPGAs), and digital signal processing units. Some of these embodiments may be entirely hardware based.

The stress profiler (1) has at least one communications interface. In this embodiment, the at least one communications interface 22 comprises a network interface in the form of an Ethernet card, however generally any suitable network interface may be used, for example a Wi-Fi module. The network interface 22 is configured, in this but not necessarily all embodiments, to send and receive information in the form of data packets. The data packets are in the form of Ethernet frames that have an Internet Protocol (IP) packet payload. The IP packets generally have a Transmission Control Protocol (TCP) segment payload, although any suitable protocol may be used. In the present embodiment, the TCP segments may carry hypertext transfer protocol (HTTP) data, for example web page information in HTTP, for example, or a HTTP request or a HTTP response. The HTTP data may be sent to a remote machine. In alternative embodiments, however, proprietary protocols and applications may be used, or generally any suitable protocol (for example SONET, Fibre Channel) or application as appropriate.

In particular, the stress profiler (1) receives stress data transmitted to it by many personal stress profilers (4) over a communications network (5) e.g. the Internet. The population stress profiler (1) also receives general data transmitted from a variety of other data sources (6), for example, news outlets, government bureaus of statistics, stock markets and weather data services.

The database (3) stores the received stress data, personal data and general data. The server (2) includes software which regularly searches for trends in the stress data, personal data and general data, and correlations between the stress, personal data and general data. In particular, the server (2) can include a learning function, which recognizes patterns of stress information associated with previous periods of stress. Over time, the learning function progressively improves the accuracy and speed of stress profiling for a user.

The server (2) can also include a predictive function which identifies patterns of stress information indicative of the early signs of stress and notify the user early. For example, the stress profiler 1 may correlate a pattern of eye movement with physiological or psychometric indicators of stress in the particular user, and notify the user when those eye movements are detected—before serious symptoms arise.

Further, the predictive function can identify patterns of stress information which are indicative of the potential for stress to arise in the future, and notify the user accordingly.

Each personal stress profiler (4) operates on a computing device such as a smart phone, smart watch, tablet computer, desktop computer or laptop, and may be wireless (as shown in FIG. 1) or use a cable connection. Each personal stress profiler can use external devices (e.g. heart rate monitors) or integrated data recording systems to make measurements and observations and uses this information to generate a stress score in each of the following forms of stress:
1. physical/physiological stress,
2. mental stress,
3. emotional stress, and
4. current perceived life stress.

Each stress score is indicative of the magnitude of a form of stress. Once a personal profiler has generated a set of stress scores, it transmits those scores plus personal data (age, location, time of measurement etc.) to the population stress profiler. However, the stress data and personal data of a user is only transmitted to the server if the user has previously consented to doing so.

As discussed above, the stress profiler (1) receives stress data and personal data from a population of people and uses the data to generate a stress profile indicative of stress experienced by the population. The stress data from each person in the population comprises at least two of:
psychometric data indicative of stress in the person,
physiological data indicative of stress in the person,
behavioural data indicative of stress in the person, and
cognitive function data indicative of stress in the person.

Receiving at least two types of stress data from each person in the population is important for a number of reasons:
1. Multiple types of stress data increases the sensitivity to lower stress levels during testing. Some forms of stress testing tend to be more sensitive to acute stress and some tend to be more sensitive to chronic stress. For example, if only physiological data are measured, then chronic stress may not be identified at all.
2. Multiple types of stress data increases the percentage of people (or ‘range’ of people) in which stress can be detected during testing. This is because stress manifests differently in different people, depending many factors such as genetic makeup, fitness, constitution and health history. Multiple types of stress testing detects more manifestations of stress.
3. Multiple types of stress data allows more specific forms of stress being experienced by people to be identified, such as acute stress or chronic stress, or other classifications, such as physical/physiological stress, mental stress, emotional stress, or current perceived life stress. The ability to identify the specific form of stress enables treatments to be prescribed which are more targeted and effective.

The population stress profiler of the present invention can be used to measure stress in large populations, for example thousands, millions or billions of people. With large numbers of people submitting the stress data and personal data, the stress profiler will receive frequent stress measurements, which enables rapid monitoring of stress to be possible.

The people in the population generate the stress data by doing standardized self-administered stress tests. Preferably, each person in the population uses a device to guide them through the self-administered stress tests and transmit both the stress data and personal data to the stress profiler. An example of such a device is the personal stress profiler described in the applicant's separate patent application filed on 11 Nov. 2014, namely Australian patent application no. 2014904524. People are motivated to use the device because it gives them direct personal feedback about their own stresses which helps to manage stress.

### The Stress Data

The amount of stress data received from people in the population will vary from person to person, depending on how much data they choose to collect and how much data they choose to share. At a minimum, the stress profiler receives two of the types of stress data from each person in the population. In one embodiment, the stress profiler receives psychometric and physiological data. However, the accuracy and sensitivity of the stress data from each person generally increases when more types of stress data are received from each person. The stress profiler may therefore receive three of the four, or all four of the types of stress data from people in the population.

The stress data is in a standard format and requires the same type of testing be used for all people in the population so that fair comparisons of the data can be made between people.

The stress data may be the raw data from each test, or it may be derivative data which is indicative of the test results, for example a test score. It is advantageous to receive a test score instead of the raw data as it reduces the amount of data to be transmitted.

### The Personal Data

Examples of the personal data that may be received by the stress profiler from people in the population include:
date of birth;
place of birth;
gender;
ethnicity;
occupation;
postal or zip code of home address;
postal or zip code of place of employment;
education;
previous postal or zip codes;
health insurance coverage;
relationship status;
number of children;
pets;
exercise and eating habits;
health history;
stress management methods currently being used.

The stress profiler uses the personal data to segment the stress data, for example by age, geographical location, occupation, relationship status, or exercise habits. The personal data helps to understand whether stress intervention methods are useful for everyone or more useful for particular segments of the population.

The stress profiler may protect privacy of users by avoiding the collection of any information which explicitly identifies the person supplying the personal and stress data.

The amount of personal data received from people in the population will vary from person to person, depending on how much data they choose to collect and how much data they choose to share.

Combining the Stress Data and Personal Data with More General Data

The stress profiler can also be arranged to receive and process many other types of general data about circumstances or events that have the potential to affect large numbers of people. The stress profiler can be arranged to search for correlations between the general data, stress data and personal data. By collecting and processing the general data, stress data and personal data, the stress profiler has the opportunity to identify causes of stress and correlations between stress and aspects of the general data. If sufficient stress data is received to monitor stress in near real time, it may be possible to use the timing of the general data and stress data to identify correlations between the two. For example, the stress profiler could monitor the effects of published news and public announcements on stress levels.

Examples of general data that may be received by the stress profiler include information indicative of:
internet keyword search behaviour;
content, sentiment or topics of social media communications;
date, time and public holidays;
temperature, humidity, weather;
traffic;
news and current affairs;
consumer purchasing data (units sold, purchase order ratings or indices, consumer confidence ratings etc.);
financial market data (currency exchange rates, commodities, shares, financial indices etc.);
economic data;
public and political announcements;
political events, sporting events and other topical events;
home loan interest rates, housing and employment data;
surveys or polls of populations;
voting schedules;
business confidence data;
business investment data;
business productivity data.

Many other types of general data can be received and processed by the stress profiler. For example, the population stress profiler can search for and identify correlations between population stress levels and usage of particular keyword search terms in Internet search engines.

### Measuring Stress Fluctuations in a Population

The data received by the population stress profiler can be used to measure fluctuations in stress in the population as a whole and segments of the population e.g. a change in stress within a particular geographic location, age, type of employment etc. With sufficient numbers of people (thousands or millions) using their personal stress profiler to submit data, the population stress profiler will be sensitive to momentary fluctuations in stress and able to monitor stresses in almost real time.

With the input of general data, the population stress profiler will be able to determine the influence of variables such as weather, news or traffic on stress levels. Stress fluctuations can be segmented according to age, gender, occupation, income, and any number of other classifications.

### Stress Index

The population stress profiler can generate a population stress index which is indicative of the magnitude of stress in the population. The population stress index can be published to show the effects of news and public announcements on stress levels.

The population stress profiler does not necessarily determine the reasons for a change in stress within a population. Rather it provides the data to show that stress went up or down on average, which provides an opportunity to investigate causes.

### Transmitting Data Back to Users

The population stress profiler can also transmit data back to the personal stress profilers.

### a) Algorithms

The population stress profiler can transmit updates to the algorithms used by the personal stress profilers to calculate stress scores.

### b) Current Population Stress Levels

The population stress profiler can transmit information about stress currently being measured in the population or a segment of the population relevant to the user of a population stress profiler. For example, the population stress profiler can inform a user about stress levels within the local area of the user, or stress levels within the same country and employment industry as the user. This type of feedback will be useful to users and may encourage users to submit their stress data and personal to the population stress profiler.
For example, if stress scores in San Francisco go up 2% then users can be informed of this so they can understand their own stress scores in that context. This improves the relevance of the stress scores measured by personal stress profilers.
The moment-to-moment data gathered by the population stress profiler improves the ability of the personal stress profilers to detect and quantify acute stress compared to chronic stress in each individual. Acute stressors are considered largely less harmful and concerning than chronic stressors, so being able to discern the difference helps to detect the type of stress that the user should be more concerned about.
It is expected that the ability of individuals to compare their scores in near real time with other comparable individuals will help to motivate people to make positive changes in relation to stress-related behaviour. Comparing oneself to others can be motivational and the near real-time nature of the information generated by the population stress profiler provides for a much greater perceived relevance.
For example, an accountant will be able to see how at tax time his counterparts are all increasing their stress scores by x %, but due to his stress management habits he is only affected by y %. He will be able to see that by improving his stress scores by a % he has, according to published research, improved his output capacity by b %.

### c) Risk Index

Over time, the population stress profiler can identify circumstances commonly associated with stress, and generate a risk index for generic circumstances. If the stress profiler has information about the personal circumstances of users, it can notify users of their own risk of experiencing higher stress, even before they report any changes in stress.
Users can also use the stress index to assist with making decisions and potentially avoid stressful situations in the future. For example, for a divorced male accountant with two children, aged 40 about to move to London and earn £70,000 per year, the population stress profiler can provide a stress index indicative of stress levels likely to be experienced in those circumstances. The accountant can take this information into account when deciding whether or not to go ahead with the move to London.
Once the user submits stress data and personal data to the population stress profiler, it can advise how their stress scores are likely to change in the future i.e. a ‘stress trajectory’. The user can use this information to implement stress management interventions and discern the likely effects these will have on stress. As the user submits further stress data and personal data, their stress trajectory will be updated.
On a much larger scale, the population stress profiler can generate a risk index and stress trajectory for a whole segment of the population, such as a whole city or country.

### The Psychometric Data

The psychometric data is indicative of responses to a questionnaire about a person's subjective experience of stress.

Preferably, the questionnaire asks questions about a wide range of signs or symptoms associated with the human stress response, particularly those aspects that are connected to the accumulation of chronic stress.

It is desirable for there to be a wide range of questions in the questionnaire so that stress can be detected in more people.

To best obtain a psychometric stress measure a ‘long-form’ and ‘short form’ questionnaire has been developed as part of this invention. In use, the psychometric stress measure will be deployed in a two stage approach, which incorporate both the ‘long form’ and the ‘short form’ questionnaires. During the first stage, an initial set of questions are posed to the individual. In a preferred embodiment, the questions that form part of this first stage will take approximately three minutes for the individual to complete. If the individual scores above a certain cut-off level, or in pre-set patterns, then the individual will be prompted to complete another block of questions, which constitutes the second stage of the questionnaire. In a preferred embodiment, this second set of questions will take approximately four to five minutes to complete. It is also envisaged that the individual will have the option (if desired) to complete the second stage set of questions, no matter their score when completing the first stage of questions.

The greater the number and severity of chronic stress indicators in the questionnaire increases the probability that they are linked to a singular underlying cause (chronic stress) rather than just occurring in the same person coincidentally. For example, one person might experience occasional tight shoulders, digestive issues and a rash that comes and goes. These symptoms, individually or even all three together, could be occurring for a number of different reasons and have nothing to do with a person developing chronic stress. However, if they also had persistent headaches, difficulty getting to sleep at night and frequent viral infections, it is beginning to tell a different story: they now have six indicators of chronic stress.

The answers to some questions may correlate strongly with other questions, forming statistically coherent factors (determined through a psychometric statistical method called Exploratory Factor Analysis). Each statistically coherent factor may be indicative of a particular type of stress being experienced by an individual.

In one embodiment, the psychometric data comprises responses to a questionnaire which asks individuals about their subjective experience of stress-related signs, symptoms or indicators across four forms of stress:
physical/physiological stress,
mental stress,
emotional stress, and
current perceived life stress.

The questionnaire can use multiple lines of questioning to cover the range of known subjective states associated with stress—particularly those noted to be indicative of chronic stress in humans. The questionnaire indicates which form of stress an individual scores more highly in. The person can then be given feedback about which type of intervention(s) are most likely to produce the greatest benefit for the person and track the results over time.

By combining the psychometric data with other types of stress data, such as physiological, behavioural or cognitive function data, the sensitivity and range to of the stress profiler is increased. Also, the other types of stress data help to detect those people who do not respond well to questionnaires.

### The Physiological Data

There are many known physiological indicators of stress in humans. Many lie detectors are based on measuring multiple physiological indicators of stress.

Where physiological information is used by the stress profiler 1, the accuracy and sensitivity of the stress profiler 1 generally increases when the physiological information includes measurements of more than one physiological parameter.

Examples of different measurements which may be used to provide physiological information include heart rate measurements, heart rate variability measurements, respiratory rate measurements, respiratory rate variability measurements, blood pressure measurements, physical movement observations, cortisol level measurements (measured in blood or saliva), skin conductivity measurements, skin temperature measurements, skin or hair analysis, DNA analysis, blood oxygen saturation measurements, surface electromyography (surface EMG) measurements, electroencephalography (EEG) measurements and measurements other physiological indicators of stress able to be determined by analysis of a person's blood, saliva or urine. The saliva, blood, urine, skin, hair and DNA measurements can be carried out through conventional laboratory testing or via nanotechnology, where for example, nanotechnology sensors can be used for single-blood drop measures, can be incorporated in a transdermal patch, can be injected subcutaneously or circulate within the body of the individual or may incorporate the use of a subcutaneously embedded microchip or wire-enabled sensor.

Furthermore, ‘smart clothing’ can also be utilised, which can include pants/trousers, underwear, socks, shoes, shirts/T-shirts, gloves, hats/caps/helmets, glasses, watches, smart-watches, wrist and ankle bands, as well as adhesive patches. The ‘smart clothing’ is embedded with various sensors, including electrical signal, conductivity (galvanic conductance and resistance), accelerometers, force, temperature, chemical sensors and nanotechnology sensors can be used to provide physiological information.

The physiological measurements may be selected in accordance with their sensitivity and relevance as well as their ease of application as a screening device.

### Physiological Data Collection Tools

The stress profiler 1 includes the ability to accept input from multiple physiological information collection tools. Each physiological information collection tool measures an aspect of the user's physiology which is indicative of stress in the user. Examples of suitable physiological information collection tools which can be used in the stress profiler 1 include, but are not limited to:
heart rate monitor, such as chest-mounted or arm-mounted devices used in sports e.g. Catapult Sports™ performance monitoring device, Polar™ heart rate monitor, Fitbit™, or smart watch capable of detecting heart rate;
respiratory rate monitor, such as chest-mounted or arm-mounted devices used in sports e.g. Catapult Sports™ performance monitoring device;
blood pressure monitor, such as a cuff around the upper arm which inflates and deflates periodically;
physical movement sensor, such as a gyroscope-enabled movement sensor used by sports people e.g. by Catapult Sports™;
location tracking device, such as a GPS-enabled smart phone or smart watch;
salivary cortisol analysis device;
skin conductivity measurement device;
skin temperature measurement device;
blood oxygen saturation measurement device e.g. finger-based pulse oximeter;
surface electromyography (surface EMG) device;
electroencephalography (EEG) device;
‘smart clothing’, including pants/trousers, underwear, socks, shoes, shirts/T-shirts, gloves, hats/caps/helmets, glasses, watches, smart-watches, wrist and ankle bands, as well as adhesive patches, embedded with various sensors, including electrical signal, conductivity (galvanic conductance and resistance), accelerometers, force, temperature, chemical sensors and nanotechnology sensors can be used to provide physiological information;
Nanotechnology sensors, which can include single-blood drop devices, transdermal patches, subcutaneous or circulatory injectable devices;
blood testing apparatus (e.g. suitable for detecting chemicals, molecules, proteins and hormones indicative of stress or stimulation of the hypothalamo-pituitary-adrenal axis (the HPA Axis) such as catecholamines, epinephrine (adrenalin), norepinephrine (noradrenaline), serotonin, or dopamine); and
human-implanted chip or wires (e.g. suitable for detecting chemicals, molecules, proteins and hormones indicative of stress or stimulation of the hypothalamo-pituitary-adrenal axis (the HPA Axis) such as catecholamines, epinephrine (adrenalin), norepinephrine (noradrenaline), serotonin, or dopamine).

The tools may be either integrated into the computing device, online or a standalone external device. Where a tool is external, it can be connected to the computing device by any suitable method, such as by cable or a wireless Bluetooth connection.

### The Behavioural Data

Where behavioural information is used by the stress profiler 1, the accuracy and sensitivity of the stress profiler 1 generally increases when the behavioural information includes measurements of more than one behavioural parameter. These behaviours may be generally known to be indicative of stress in humans, or they may be individual traits of the user. For example, a user may exhibit a particular pattern of eye movement, pace up and down, or visit a particular location when stressed.

The stress profiler 1 may progressively acquire behavioural information by progressively correlating behaviours with other forms of stress information, such as cognitive function information, psychometric information or physiological information.

Examples of different measurements or behavioural observations which may be used to provide behavioural information include eye movement patterns, social interactions, the types of websites visited, the types of apps used, the news topics read, spending behaviour, food choices, social outings, taking holidays, and so on.

Data can be obtained from smartphones, smart-watches or other wearable devices, tablets and computers, which can be measured by the accelerometer, gyroscope, altimeter, GPS, NFC (proximity to other devices, enhanced location specificity), Bluetooth (proximity to other devices, enhanced location specificity), Wi-Fi (proximity to other devices, enhanced location specificity). Other inputs can be measured such as, keystroke rate, cadence, typing style, pressure or ‘force’ detection (keypad, trackpad, screen pressure sensor), voice analysis (tone, cadence, word and phrase detection), phone usage, including call time, numbers dialed, time of day calls placed, Application (‘app’) usage, including specific applications used, duration of usage, time of day apps used, in-app analytics (use characteristics within any app), keyword searches, word and phrase usage (usually applied within word processing, email, messaging and social media applications but not limited to these), eye movement patterns, gait and posture analysis and purchasing history.

Other behavioural observations can be obtained from car/driving/riding style, which include steering inputs, acceleration, deceleration, braking, speed of driving, brake and accelerator force, door pressure sensors and other vehicle sensors.

Further behavioural observations can be obtained from home or office sensors, which can measure movement, body temperature, television usage (channels watched, time watching, eye movement), refrigerator analytics, heating and cooling analytics and other ‘smart home’ analytics.

Additionally, behavioural observations can also be obtained from other measurement devices such as bicycle meters (pedal force, pedaling cadence, acceleration, speed, routes taken, GPS, altimeter, time on bicycle, and so on), pedometers, gait analysis measures and other measurements obtained from ‘smart clothing’, which includes pants/trousers, underwear, socks, shoes, shirts/T-shirts, gloves, hats/caps/helmets, glasses, watches, smart-watches, wrist and ankle bands, as well as adhesive patches.

### Behavioural Data Collection Tools

The stress profiler 1 includes the ability to accept input from multiple behavioural information collection tools. Each behavioural information collection tool measures an aspect of the user's behaviour which is indicative of stress in the user. Examples of suitable behavioural information collection tools which can be used in the stress profiler 1 include, but are not limited to:
eye-tracking software;
a location tracking device, such as a GPS-enabled smart phone or smart watch;
Bluetooth tracking software to track the nearby presence of devices owned by other individuals;
internet browsing history analysis software;
smartphone, smart-watch or other wearable device, tablet or computer accelerometers, gyroscopes or altimeters,
proximity sensing devices such as NFC, Wi-Fi or Bluetooth, particularly with enhanced location specificity, (proximity to other devices, enhanced location specificity),
keystroke rate, cadence, typing style, pressure or ‘force’ detection (keypad, trackpad, screen pressure sensor);
voice analysis (tone, cadence, word and phrase detection), phone usage, including call time, numbers dialed, time of day calls placed,
application (‘app’) usage, including specific applications used, duration of usage, time of day apps used, in-app analytics (use characteristics within any app), keyword searches, word and phrase usage (usually applied within word processing, email, messaging and social media applications but not limited to these), gait and posture analysis and purchasing history;
car/driving/riding style, including steering inputs, acceleration, deceleration, braking, speed of driving, brake and accelerator force, door pressure sensors and other vehicle sensors;
home or office sensors, which can measure movement, body temperature, television usage (channels watched, time watching, eye movement), refrigerator analytics, heating and cooling analytics and other ‘smart home’ analytics;
bicycle meters (pedal force, pedaling cadence, acceleration, speed, routes taken, GPS, altimeter, time on bicycle, and so on), pedometers, gait analysis measures; and
‘smart clothing’, which includes pants/trousers, underwear, socks, shoes, shirts/T-shirts, gloves, hats/caps/helmets, glasses, watches, smart-watches, wrist and ankle bands, as well as adhesive patches

The stress profiler 1 first requests permission from the user to collect behavioural information, and then routinely collects the information in the background without interrupting the user.

The tools may be either integrated into the computing device, online or a standalone external device. Where a tool is external, it can be connected to the computing device by any suitable method, such as by cable or a wireless Bluetooth connection.

### The Cognitive Function Data

The cognitive function data is indicative of stress-related cognitive function measurements made on people in the population.

Examples of cognitive function measurements include the results of memory tests, reaction-time measurements, and the results of decision-making tests. The accuracy and sensitivity of cognitive function measurements generally increases when more than one cognitive function parameter are measured.

The cognitive function or performance tests can be in the form of online tasks, or interaction with smart watches, smart phones or other computing devices.

There is literature on the correlation between cognitive function and stress in humans, for example: “Stress Effects on Working Memory, Explicit Memory, and Implicit Memory for Neutral and Emotional Stimuli in Healthy Men”, Mathias Luethi, Beat Meier, Carmen Sandi, Frontiers of Behavioural Neuroscience, 2008; 2: 5

### Cognitive Function Data Collection Tools

The stress profiler 1 includes the ability to accept input from multiple cognitive function information collection tools. Each cognitive function information collection tool measures an aspect of the user's cognitive function which is indicative of stress in the user. Examples of suitable cognitive function information collection tools which can be used in the stress profiler 1 include, but are not limited to:
software to test the memory of a user;
software to test the reaction time of a user;
software to test the attention, peripheral vision and comprehension of a user;
software to test the decision-making ability of a user.

The processor prompts the user to complete one or more of the cognitive function tests. If the user agrees to do the test(s), the processor presents the user with a brief cognitive function test. The test should generally be quick to do, and perhaps take from 5 seconds to 2 minutes to complete. The memory test may prompt the user at a later time to remember a piece of information.

The tools may be either integrated into the computing device, online or a standalone external device. Where a tool is external, it can be connected to the computing device by any suitable method, such as by cable or a wireless Bluetooth connection.

### EXAMPLES

### Embodiment 1

This embodiment is a mobile version of the stress profiler 1 in which each of the individuals of the population, in this Example within a relatively small geographical area, operate a smart phone, smart watch or tablet computing device to provide the relevant individual stress information.

In particular, the devices utilised by each of the plurality of people in the population include a mobile app. Some of the relevant stress information is collected by the app in the background without any manual input by the user, and the remainder of the information requires active participation of the user.

As disclosed above, preferably, each person in the population uses the device to guide them through self-administered stress tests and transmits both the stress data and personal data to the stress profiler. An example of such a device is the personal stress profiler described in the applicant's separate patent application filed on 11 Nov. 2014, namely Australian patent application no. 2014904524. In this way, an individual's stress rating is calculated using a smartphone, desktop computer, tablet or any other suitable connected device, such as smart watch, smart clothing, nanotechnology sensor, etc.

Once calculated, this score is transmitted to the central server bank via conventional communication channels (as available), such as Wi-Fi, mobile or satellite connection and/or via the Internet, and the score is collated with previously recorded data shared by the user (demographic, gender, occupation, lifestyle, etc.). Other user's physical stress ratings are similarly collated by the central servers and the aggregate physical stress data from multiple users is used to calculate a group or population aggregate physical stress score average:

Population x stress score (can be categorized or defined by geographical location, gender, occupation, age and so on, or refined subcategories) over a specified time period (minutes, hours, days, weeks, months or years)=
a) user a) physical stress score over the specified time period+
b) user b) physical stress score over the specified time period+
c) user c) physical stress score over the specified time period+ . . .
. . . and so on for of the number of people within the relevant population.

Divided by the total number of included users (i.e. individuals) in the population (a+b+c . . . /number of users included in the total) in the specified time period=Population×physical stress score for the specified time period.

As an example of the above, one such population for which the system and method for generating a profile of stress levels and stress resilience levels of the present invention can be utilised is a discrete geographic location of Cambridge, Mass. in the United States of America. In particular, the population of relevance for this particular Example is that of the suburb comprising the Harvard University campuses.

The population physical stress measure or score for Cambridge, Mass. would comprise the physical stress scores of all active users (i.e. each one of the plurality of individuals within the population) in this suburb. The population physical stress measure or score is measured constantly throughout every day using the connected devices listed above, i.e. smartphones, tablets, desktop computers, smart watches, etc. The relevant data is transmitted to the central servers via conventional communication channels, such as Wi-Fi, mobile communication networks or other means via the Internet. These physical stress scores may be a very accurate measure of acute or short-term stress in particular.

Typically, it is expected that the average physical stress score for the whole of this population in Cambridge, Mass. would rise at the beginning of the academic year, and again around exam times and/or immediately prior to the end of semester.

Typically, it is expected that this average physical stress scores would then drop significantly as the summer vacation break commences.

Within the scope of this invention, it is possible to further refine the population for Cambridge, Mass. to only include people aged 17 to 28 years of age. With this ‘sub-population’ of younger people (who would likely be students), it would be expected that the data would provide even greater physical stress scores than the average over these time periods.

Similarly, if a ‘sub-population’ of academic professionals, such as professors and support staff, was selected it would be expected that a different ‘population pattern’ of stress would be displayed, most likely showing an elevation at the beginning of the academic year, but lower than usual around exam times when the workload of most academic professionals would be reduced, and then elevated again immediately following exam times when there is significant pressure on the academic professionals to grade results.

These varying population stress levels could inform the policy of the university to institute stress management initiatives directed towards the specific sub-populations at the times they are most needed, enabling better support of students and staff as well as a more refined use of resources.

Published ‘population×physical stress score for the specified time period’ scores may also be weighted by multiplying the individual or ‘population×physical stress score for the specified time period’ by a weighting coefficient to accommodate idiosyncrasies or variations in populations or to account for the influence of particular variables such seasonal changes and the like in order to make comparisons more accurate and or useful.

To continue the Cambridge, Mass. example above, this is a particular geographic location that experiences extreme cold in winter. This may cause physical stress scores to rise independently of any workplace-related stressors during the cold winter months—and even more particularly in the event of an unusually cold winter, an excessively prolonged winter, a ‘once in a lifetime’ blizzard/storm or the like. In order to discern accurate stress levels due to workplace stress and the desirability or necessity of interventions, the effect of the weather would need to be accommodated by a weighting coefficient; elevating physical stress scores in a population through a period of particularly foul weather would not necessarily warrant concern or intervention by the employer.

As another example of this ‘weighting’, consider a geographic location subject to high variance in population due to ‘fruit picking’: the influx of seasonal workers with their own individual physical stress characteristics might influence the average physical stress score for that location. A ‘seasonally adjusted physical stress score’ may provide more useful data for an individual considering moving to that location permanently or for the calculation of the provision of health services or in calculating the influence of political announcements on overall stress levels.

This ‘population×physical stress score for the specified time period’ can then be correlated with other data related to traffic, weather, political announcements, news, and the like to determine the influence of external and environmental events on the stress levels of whole populations or sub-populations.

Again continuing the Cambridge, Mass. example above. If there was a political announcement that a heavy polluting industry had received approval to dump millions of tonnes of toxic materials every year into the Charles River immediately upstream of Boston, it might be expected that the inhabitants of the Boston area would become upset or stressed.

Witnessing this stress level increase in possibly a million people or more, and its link to the political announcement could be very beneficial on several fronts. The management of Harvard University, MIT and Boston College could then be able to understand stress levels in their staff and possibly students and accommodate this as an influencing stressor not caused by the university workload. The government would also have data to show the likely reduced productivity as a result of stress and likely increased health care expense as a result of stress for the Boston area and as a result this information could provide tangible data for governments to incorporate into their decision-making processes that was unavailable before now; productivity losses and increased healthcare expense throughout the region might outweigh the economic benefit of the new industry.

Variations and/or modifications may be made to the embodiments described without departing from the spirit or ambit of the invention. The present embodiments are, therefore, to be considered in all respects as illustrative and not restrictive.

Prior art, if any, described herein is not to be taken as an admission that the prior art forms part of the common general knowledge in any jurisdiction.

In the claims which follow and in the preceding description of the invention, except where the context requires otherwise due to express language or necessary implication, the word “comprise” or variations such as “comprises” or “comprising” is used in an inclusive sense, that is to specify the presence of the stated features but not to preclude the presence or addition of further features in various embodiments of the invention.

1. A method for generating stress level information indicative of a stress level of a plurality of individuals, the method comprising:
receiving, via a network, individual stress information for each of the plurality of individuals; and
generating, in a processing system, a statistical value for the stress level of the plurality of individuals by statistically processing the individual stress information for each of the plurality of individuals.

2. A method defined by claim 1 comprising the steps of:
receiving, via the network, personal information for each of a plurality of persons and individual stress information for each of the plurality of persons
in the processing system, using personal information for each of the plurality of persons to select from the plurality of persons the plurality of individuals.

3. A method defined by claim 2 wherein the personal information comprises at least one of date of birth information, place of birth information, gender information, ethnicity information, occupation information, postcode information, education information, health insurance coverage information, relationship status information, number of children information, pet information, exercise habit information, eating habit information, health history information, and information indicative of stress management methods currently being used.

4. A method defined by claim 1 comprising the step of receiving, via the network, information indicative of at least one of a stress modifying circumstance and stress modifying event and correlating a stress feature in the statistical measure with the at least one of the stress modifying circumstance and the stress modifying event.

5. A method defined by claim 4 wherein the stress feature comprises a change in the statistical value of the stress level of the plurality of individuals.

6. A method defined by claim 4 wherein the at least one of the stress modifying circumstance and the stress modifying event comprises at least one of: internet keyword search behaviour information, content information, sentiment or topics of social media communications information, date information, time information, public holiday information, temperature information, humidity information, weather information, traffic information, news information, current affairs information, consumer purchasing information, financial market information, economic information, announcement information, political event information, sporting event information, topical event information, home loan interest rate information, housing information, employment information, survey information, poll information, voting schedule information, business confidence information, business investment information, and business productivity information.

7. A method defined by claim 1 comprising the step of generating, in the processing system, a stress index using the statistical value.

8. A method defined by claim 7 comprising the step of the processing system sending the stress index to a plurality of computing devices.

9. A method defined by claim 8 comprising the step of the processing system sending the statistical measure of the stress level of the plurality of individuals to the plurality of computing devices.

10. A method defined by claim 1 wherein the individual stress information for each of the plurality of individuals comprises at least one of psychometric information for each of the plurality of individuals, physiological information for each of the plurality of individuals, behavioural information for each of the plurality of individuals, and cognitive function information for each of the plurality of individuals.

11. A method defined by claim 10 wherein the individual stress information for each of the plurality of individuals comprises at least two of psychometric information for each of the plurality of individuals, physiological information for each of the plurality of individuals, behavioural information for each of the plurality of individuals, and cognitive function information for each of the plurality of individuals.

12. A method defined by claim 10 wherein the stress information for each of the plurality of individuals comprises the psychometric information for each of the plurality of individuals.

13. A method defined by claim 12 comprising the step of generating the psychometric information for each of the plurality of individuals by each of the plurality of individuals responding to an electronic stress questionnaire.

14. A method defined by claim 13 wherein the questionnaire is in two parts, each comprising a different set of predefined questions, whereby the individual is presented with the second set of questions based on predetermined criteria correlating with the answers provided to the first set of questions.

15. A method defined by claim 12 wherein the psychometric information for each of the plurality of individuals is indicative of a plurality of chronic stress indicators for the each of the plurality of individuals.

16. A method defined by claim 10 wherein the stress information for each of the plurality of individuals comprises the physiological information for each of the plurality of individuals.

17. A method defined by claim 16 comprising the step of generating the physiological information for each of the plurality of individuals.

18. A method defined by claim 17 wherein the step of generating the physiological information for each of the plurality of individuals comprises the step of generating information for each of a plurality of physiological functions in each of the plurality of individuals.

19. A method defined by claim 18 wherein the step of generating the physiological information for each of the plurality of individuals comprises the step of generating at least one of heart rate information, heart rate variability information, respiratory rate information, respiratory rate variability information, blood pressure information, physical movement information, cortisol level information, skin conductivity information, skin temperature information, skin or hair analysis, DNA analysis, blood oxygen saturation information, surface electromyography information, electroencephalography information, blood information, saliva information, skin conductance information, information regarding the chemicals found on or within the skin, and urine information.

20. A method defined by claim 10 wherein the stress information for each of the plurality of individuals comprises behavioural information for each of the plurality of individuals.

21. A method defined by claim 20 comprising the step of generating the behavioural information for each of the plurality of individuals.

22. A method defined by claim 21 wherein the step of generating the behavioural information for each of the plurality of individuals comprises at least one of the steps of:
generating eye movement information indicative of eye movement of the individual;
generating location information indicative of a plurality of locations the individual has been;
generating nearby device information indicative of the nearby presence a plurality of devices of a plurality of people to the individual;
generating internet browsing history information for the individual;
generating keystroke rate, cadence, typing style, pressure or ‘force’ detection information for the individual;
generating voice analysis, including tone, cadence, word and phrase detection information for the individual;
generating telephone usage analysis, including call time, numbers dialed and time of day calls placed information for the individual;
generating driving style, including steering inputs, acceleration, deceleration, braking, speed of driving, brake and accelerator force and data from door pressure sensor information for the individual;
generating movement, body temperature, television usage, including channels watched, time watched and eye movement whilst watching, refrigerator analytics, heating and cooling analytics information for the individual;
generating bicycle data, including pedal force, pedaling cadence, acceleration, speed, routes taken, GPS data, altimeter data, time on bicycle, pedometer data information for the individual;
generating pedometer data and gait analysis information for the individual;
generating application usage information indicative of application usage by the individual;
generating media consumption information indicative of media consumption by the individual;
generating spending behaviour information indicative of the individual's spending behaviour;
generating food choice information indicative of a plurality of food choices made by the individual;
generating social outing information indicative of the individual's social outing activity; and
generating leave information indicative of leave taken by the individual.

23. A method defined by claim 10 wherein the stress information for each of the plurality of individuals comprises the cognitive function information for each of the plurality of individuals.

24. A method defined by claim 23 comprising the step of generating the cognitive function information for each of the plurality of individuals.

25. A method defined by claim 24 wherein the step of generating the cognitive function information for each of the plurality of individuals comprises at least one of the steps of:
generating memory function information indicative of a memory function of each of the plurality of individuals;
generating reaction time information indicative of a reaction time of each of the plurality of individuals;
generating attention ability, peripheral vision and comprehension ability of the individual; and
generating decision-making ability information indicative of a decision-making ability of each of the plurality of individuals.

26. A method defined by claim 1 further comprising a step of generating a stress resilience score indicative of each of the plurality of individuals response to acute stress.

27. A method defined by claim 26 wherein the stress resilience score is indicative of one or more of the time taken for the plurality of individuals to respond to an acute stress event, if the plurality of individuals exhibit any response to an acute stress event, and if so, the level of response exhibited by the plurality of individuals to an acute stress event and the time taken for the plurality of individuals' stress information to return to baseline levels following a period of acute stress.

28. A processing system for generating stress level information indicative of a stress level of a plurality of individuals, the system comprising:
a receiver configured to receive via a network individual stress information for each of the plurality of individuals; and
a statistical value generator configured to generate a statistical value for the stress level of the plurality of individuals by statistically processing the individual stress information for each of the plurality of individuals.
